(19) United States
(12) Patent Application Publication
(10) Pub. No.: US 2023/0035592 A1
(43) Pub. Date: Feb. 2, 2023

(54) DIRECT AMPK ACTIVATOR COMPOUNDS COMBINED WITH INDIRECT AMPK ACTIVATOR COMPOUNDS, COMPOSITIONS, METHODS AND USES THEREOF

(71) Applicant: SOCIETE DES PRODUITS NESTLE S.A., Vevey (CH)

(72) Inventors: MATTHEW SANDERS, Epalinges (CH); YANN RATINAUD, Morges (CH); DENIS MARCEL BARRON, Lutry (CH); KEI SAKAMOTO, København Ø (DK)

(21) Appl. No.: 17/756,422

(22) PCT Filed: Nov. 24, 2020
(86) PCT No.: PCT/EP2020/083115

§ 371 Date: May 25, 2022

(30) Foreign Application Priority Data
Nov. 27, 2019 (EP) 19211690.3

(51) Int. Cl. A61K 31/366; A23L 33/10

(52) U.S. Cl. CPC A61K 31/366; A23L 33/10

(57) ABSTRACT

The present invention relates to a combinations of direct AMPK activators with indirect AMPK activators for use in activating AMPK. In particular, combinations of benzocoumarins of formula I which are direct AMPK activators with urolithins of formula VII which are indirect AMPK activators.

### BACKGROUND OF THE INVENTION

AMP-activated protein kinase (AMPK) is an evolutionarily conserved master regulator of energy homeostasis that coordinates metabolic pathways in order to balance nutrient supply with energy demand. AMPK is considered a key drug target to combat the growing epidemic of metabolic disorders such as obesity, type 2 diabetes, cardiovascular disease.

AMPK activity is found in all tissues, including liver, kidney, muscle, lung, and brain (PMID: 10698692). In terms of structure, AMPK is a heterotrimeric complex consisting of a catalytic subunit (α) and two regulatory subunits (β and γ). The AMPK complex is evolutionarily conserved and also can be found in yeast and plants. Mammalian AMPK is composed of different isoforms of subunits: α1, α2, β1, β2, γ1, γ2, and γ3 (PMID: 11746230) leading to 12 possible heterotrimeric combinations. The α2 isoform is predominately found in skeletal and cardiac muscle AMPK; both the α1 and α2 isoforms are found in hepatic AMPK; while for example in adipose and T cells the al isoform AMPK predominates (PMID: 16818670, PMID 15878856).

There is no direct AMPK-activating pharmaceutical drug available to treat the various diseases and conditions where there is low AMPK activation despite intensive efforts continuously made by the pharmaceutical industry. There is not thought to be any clinical trials registered to test the effects of AMPK-activating drug. Several synthetic AMPK activators have been identified/developed. However, they either have no/poor oral availability (PMID: 16753576, PMID: 24900234) or there are concerns about their adverse effects, since chronic and strong AMPK activation may cause increases in cardiac glycogen content and hypertrophy (PMID: 11827995).

There are numerous natural compounds/extracts known to bring about some metabolic health benefits that are shown to indirectly stimulate AMPK most likely through inhibition of mitochondrial respiration. However, whether those metabolic effects are mediated by AMPK is largely elusive, and moreover there are concerns regarding side effects and toxic effects such as cellular or mitochondrial poisoning.

Natural compounds activate AMPK almost exclusively through their ability to interfere with ATP production of the cell, typically by inhibiting mitochondrial respiration. As a consequence, this perturbs the adenine nucleotide levels within the cell and leads to activation of AMPK through AMP and ADP binding to the AMPK γ subunit. This mechanism of AMPK activation has been termed “indirect” due to the fact that natural compounds do not directly bind to AMPK to achieve activation.

In contrast, AMPK can be “directly” activated by binding of molecules to the allosteric drug and metabolite (ADaM) binding site formed at the interface between the AMPK a subunit kinase domain and the AMPK β subunit (Bultot, et al., (2016) Am J Physiol Endocrinol Metab 311(4): E706-e719; Ducommun et al., (2014) Am J Physiol Endocrinol Metab 306(6): E688-696; Ford et al., (2015) Biochem J 468(1): 125-132; O'Brien et al. (2015) Biochem J 469(2): 177-187).

Advantageously, by combining both the “direct” and “indirect” activators of AMPK lower concentrations can be used to achieve a considerable activation of AMPK. This is an attractive therapeutic approach since it can reduce the cost of treatment associated with high concentrations of compounds but also help avoid side effects or potential side effects associated with using the individual AMPK activators alone.

Furthermore, natural compounds are more often poorly bioavailable and are cleared from the body due to glucoronidation, sulphonation and methylation. Therefore, these natural compounds sometimes do not accumulate in sufficiently high doses to bring about an effect when used alone in vivo, whereas, when used in combination with a synergistic AMPK activator, the low concentrations of the individual natural compounds can have efficacy.

Lastly, there can be inter-individual variability in the bioavailability or metabolism of natural compounds, which means that some individuals do not show efficacy when using a single natural compound. In particular, natural compounds that are transformed by the microbiota into active compounds, can show variability with single natural compound treatment due to variations in the gut microbiota. By advantageously combining natural compounds that are synergistic in their activation, these low inefficacious concentrations of natural compounds can display efficacy when used in combination.

Surprising, the present invention demonstrates that by combining direct activators at the ADaM site with indirect activators working indirectly to alter the nucleotide status of the cell by lowering ATP in the cell and increasing AMP/ADP, to activate AMPK via the gamma-subunit, it is possible to achieve synergistic activation of AMPK.

The present invention addresses the clear unmet need for new natural compounds and combinations to activate AMPK with the advantages mentioned above.

### SUMMARY OF THE INVENTION

The present invention relates to combinations of direct AMPK activators and indirect AMPK activators which are used for activation of AMPK.

The direct AMPK activators of the invention act by binding the ADaM binding site at the interface between the alpha-subunit of AMPK and the beta-subunit of AMPK. The indirect AMPK activators act indirectly to alter the nucleotide status of the cell by lowering ATP in the cell and increasing AMP/ADP, to activate AMPK via the gamma-subunit.

The invention relates to the direct AMPK activator compounds having the general formula (I),

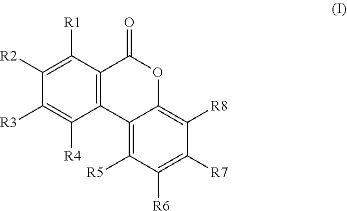

wherein R1, R2, R3, R4, R5, R6, R7, and R8 are each independently H; CH₃; CH₂OH; CHO; COOH; OH; OCH₃; CO—(CH₂)₂—CH₃; O—CO—CH₃; a halogen; O-glycoside; C-glycoside; acylated O-glycoside; acylated C-glycoside; sulfated O-glycoside; sulfated C-glycoside; a sulfate; or a derivative or analogue thereof, for use in the direct activation of AMPK.

In some embodiments, an OCH₃ group can cyclize with a neighboring OH group to form a methylene dioxy bridge.

In another embodiment, the invention relates to direct AMPK activator compounds of formula (II)

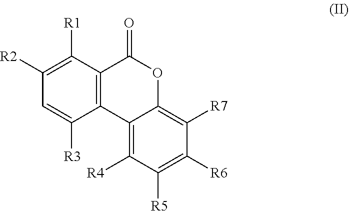

wherein R1, R2, R3, R4, and R5 are each independently OH; OCH₃; O-glycoside; C-glycoside;

acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl; R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R6, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; R3, R4, and R5 are each independently OH; OCH₃; O-glycoside; C-glycoside; acylated O-glycoside; acylated C-glycoside; sulfated O-glycoside; sulfated C-glycoside; a sulfate; R6, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK. In some embodiments, an OCH₃ group can cyclize with a neighboring OH group to form a methylene dioxy bridge.

In one embodiment, R1 and R2 are each independently OH; OCH₃; O-glycoside; C-glycoside;

acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4 and R5 are each independently OH; OCH₃; O-glycoside; C-glycoside; acylated O-glycoside; acylated C-glycoside; sulfated O-glycoside; sulfated C-glycoside; a sulfate;

R6 and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R6, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside;

C-glycoside; acylated O-glycoside; acylated C-glycoside; sulfated O-glycoside; sulfated C-glycoside; a sulfate;

R6, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R4 and R5 are each independently OH; OCH₃; O-glycoside; C-glycoside; acylated O-glycoside; acylated C-glycoside; sulfated O-glycoside; sulfated C-glycoside; a sulfate; R6 and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl; R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; R3, R6 and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; R4 and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6, and R7 are each independently OH; OCH₃; O-glycoside; C-glycoside; acylated O-glycoside; acylated C-glycoside; sulfated O-glycoside; sulfated C-glycoside; a sulfate; R4, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK. In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6, and R7 are each independently OH; OCH₃; O-glycoside; C-glycoside; acylated O-glycoside; acylated C-glycoside; sulfated O-glycoside; sulfated C-glycoside; a sulfate;

R4 and R5 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R6 and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK. In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a sulfate; R4 and R5 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5 and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5, and R6 are each independently OH; OCH₃; O-glycoside; C-glycoside; acylated O-glycoside; acylated C-glycoside; sulfated O-glycoside; sulfated C-glycoside; a sulfate;

R4, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5, and R6 are each independently OH; OCH₃; O-glycoside; C-glycoside; acylated O-glycoside; acylated C-glycoside; sulfated O-glycoside; sulfated C-glycoside; a sulfate;

R4 and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R5 and R6 are each independently OH; OCH₃; O-glycoside;

C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a sulfate;

R4, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a sulfate; R4 and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, R5, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, R5, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside;

acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, R5 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a sulfate; R4, R5, and R7 are H, or a derivative or analogue thereof, for use in the activation of AMPK.

In some embodiments, an OCH₃ group can cyclize with a neighboring OH group to form a methylene dioxy bridge.

In another embodiment, the invention relates to direct AMPK activator compounds of formula (III)

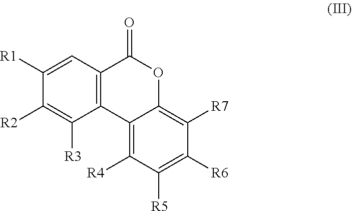

wherein R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4 and R5 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R6, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4, and R5 are each independently OH; OCH₃; O-glycoside; C-glycoside; acylated O-glycoside; acylated C-glycoside; sulfated O-glycoside; sulfated C-glycoside; a sulfate;

R6, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4 and R5 are each independently OH; OCH₃; O-glycoside; C-glycoside; acylated O-glycoside; acylated C-glycoside; sulfated O-glycoside; sulfated C-glycoside; a sulfate;

R6 and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside;

C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R6, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a sulfate;

R6, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R4 and R5 are each independently OH; OCH₃; O-glycoside; C-glycoside; acylated O-glycoside; acylated C-glycoside; sulfated O-glycoside; sulfated C-glycoside; a sulfate;

R6 and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside;

C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6 and R7 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6, and R7 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6, and R7 are each independently OH; OCH₃; O-glycoside; C-glycoside; acylated O-glycoside; acylated C-glycoside; sulfated O-glycoside; sulfated C-glycoside; a sulfate;

R4 and R5 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R6 and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a sulfate;

R4 and R5 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside;

C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside;

acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5 and R6 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5, and R6 are each independently OH; OCH₃; O-glycoside; C-glycoside; acylated O-glycoside; acylated C-glycoside; sulfated O-glycoside; sulfated C-glycoside; a sulfate;

R4, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5, and R6 are each independently OH; OCH₃; O-glycoside; C-glycoside; acylated O-glycoside; acylated C-glycoside; sulfated O-glycoside; sulfated C-glycoside; a sulfate;

R4 and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, R5 and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a sulfate;

R4, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside;

C-glycoside; acylated O-glycoside; acylated C-glycoside; sulfated O-glycoside; sulfated C-glycoside; a sulfate; R4 and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, R5, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, R5, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are H, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, R5 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are H, or a derivative or analogue thereof, for use in the activation of AMPK.

In some embodiments, an OCH₃ group can cyclize with a neighboring OH group to form a methylene dioxy bridge.

In another embodiment, the invention relates to direct AMPK activator compounds formula (IV)

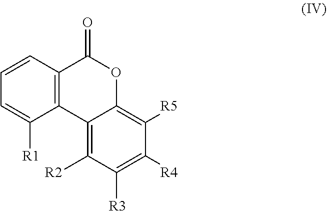

wherein R1, R2, and R3 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, and R3 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1, R2 and R3 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, and R3 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2 and R3 are each independently OH; OCH₃; O-glycoside; C-glycoside; acylated O-glycoside; acylated C-glycoside; sulfated O-glycoside; sulfated C-glycoside; a sulfate;

R4 and R5 are H, or a derivative or analogue thereof, for use in the activation of AMPK.

In some embodiments, an OCH₃ group can cyclize with a neighboring OH group to form a methylene dioxy bridge.

In one embodiment, R1, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R2, and R3 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R4 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, and R3 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1, R4 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R2, and R3 are H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R4, and R5 are each independently OH; OCH₃; O-glycoside; C-glycoside; acylated O-glycoside; acylated C-glycoside; sulfated O-glycoside; sulfated C-glycoside; a sulfate;

R2, and R3 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R4, and R5 are each independently OH; OCH₃; O-glycoside; C-glycoside; acylated O-glycoside; acylated C-glycoside; sulfated O-glycoside; sulfated C-glycoside; a sulfate;

R2 and R3 are H, or a derivative or analogue thereof, for use in the activation of AMPK.

In some embodiments, an OCH₃ group can cyclize with a neighboring OH group to form a methylene dioxy bridge.

In one embodiment, R1, R3, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R2, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R3, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1, R3 and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R2, and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R3, and R4 are each independently OH; OCH₃; O-glycoside; C-glycoside; acylated O-glycoside; acylated C-glycoside; sulfated O-glycoside; sulfated C-glycoside; a sulfate;

R2, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R3, and R4 are each independently OH; OCH₃; O-glycoside; C-glycoside; acylated O-glycoside; acylated C-glycoside; sulfated O-glycoside; sulfated C-glycoside; a sulfate;

R2 and R5 are H, or a derivative or analogue thereof, for use in the activation of AMPK.

In some embodiments, an OCH₃ group can cyclize with a neighboring OH group to form a methylene dioxy bridge.

In one embodiment, R1, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro;

a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R2, R3, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R3, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R2, R3, and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 and R4 are each independently OH; OCH₃; O-glycoside; C-glycoside; acylated O-glycoside; acylated C-glycoside; sulfated O-glycoside; sulfated C-glycoside; a sulfate;

R2, R3, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 and R4 are each independently OH; OCH₃; O-glycoside; C-glycoside; acylated O-glycoside; acylated C-glycoside; sulfated O-glycoside; sulfated C-glycoside; a sulfate;

R2, R3, and R5 are H, or a derivative or analogue thereof, for use in the activation of AMPK.

In another embodiment, the invention relates to direct AMPK activator compounds of formula (V)

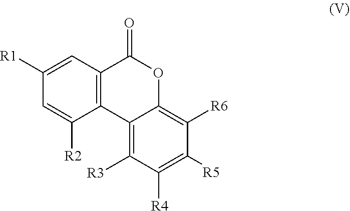

wherein R1, R2, R3, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R5 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R5 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R3 and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R5, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside;

acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R3, and R4 are each independently OH; OCH₃; O-glycoside; C-glycoside; acylated O-glycoside; acylated C-glycoside; sulfated O-glycoside; sulfated C-glycoside; a sulfate;

R5, and R6 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R3 and R4 are each independently OH; OCH₃; O-glycoside; C-glycoside; acylated O-glycoside; acylated C-glycoside; sulfated O-glycoside; sulfated C-glycoside; a sulfate;

R5 and R6 are H, or a derivative or analogue thereof, for use in the activation of AMPK.

In some embodiments, an OCH₃ group can cyclize with a neighboring OH group to form a methylene dioxy bridge.

In one embodiment, R1, R2, R3 and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R5, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, and R4 are each independently OH; OCH₃; O-glycoside; C-glycoside; acylated O-glycoside; acylated C-glycoside; sulfated O-glycoside; sulfated C-glycoside; a sulfate;

R5, and R6 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3 and R4 are each independently OH; OCH₃; O-glycoside; C-glycoside; acylated O-glycoside; acylated C-glycoside; sulfated O-glycoside; sulfated C-glycoside; a sulfate;

R5 and R6 are H, or a derivative or analogue thereof, for use in the activation of AMPK.

In some embodiments, an OCH₃ group can cyclize with a neighboring OH group to form a methylene dioxy bridge.

In one embodiment, R1, R2, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

R3 and R4 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R4 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R5 and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3 and R4 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R5, and R6 are each independently OH; OCH₃; O-glycoside; C-glycoside; acylated O-glycoside; acylated C-glycoside; sulfated O-glycoside; sulfated C-glycoside; a sulfate;

R3, and R4 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R5, and R6 are each independently OH; OCH₃; O-glycoside; C-glycoside; acylated O-glycoside; acylated C-glycoside; sulfated O-glycoside; sulfated C-glycoside; a sulfate;

R3 and R4 are H, or a derivative or analogue thereof, for use in the activation of AMPK.

In some embodiments, an OCH₃ group can cyclize with a neighboring OH group to form a methylene dioxy bridge.

In one embodiment, R1, R2, R5, and R6 are each independently OH; OCH₃; O-glycoside; C-glycoside; acylated O-glycoside; acylated C-glycoside; sulfated O-glycoside; sulfated C-glycoside; a sulfate;

R3, and R4 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R5, and R6 are each independently OH; OCH₃; O-glycoside; C-glycoside; acylated O-glycoside; acylated C-glycoside; sulfated O-glycoside; sulfated C-glycoside; a sulfate;

R3 and R4 are H,

or a derivative or analogue thereof, for use in the activation of AMPK.

In some embodiments, an OCH₃ group can cyclize with a neighboring OH group to form a methylene dioxy bridge.

In one embodiment, R1, R2, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R3 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R4 and R5 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R4, and R5 are each independently OH; OCH₃; O-glycoside; C-glycoside; acylated O-glycoside; acylated C-glycoside; sulfated O-glycoside; sulfated C-glycoside; a sulfate;

R3, and R6 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R4, and R5 are each independently OH; OCH₃; O-glycoside; C-glycoside; acylated O-glycoside; acylated C-glycoside; sulfated O-glycoside; sulfated C-glycoside; a sulfate;

R3 and R6 are H, or a derivative or analogue thereof, for use in the activation of AMPK.

In some embodiments, an OCH₃ group can cyclize with a neighboring OH group to form a methylene dioxy bridge.

In one embodiment, R1, R2, R4 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R4, and R5 are each independently OH; OCH₃; O-glycoside; C-glycoside; acylated O-glycoside; acylated C-glycoside; sulfated O-glycoside; sulfated C-glycoside; a sulfate;

R3, and R6 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R4, and R5 are each independently OH; OCH₃; O-glycoside; C-glycoside; acylated O-glycoside; acylated C-glycoside; sulfated O-glycoside; sulfated C-glycoside; a sulfate;

R3 and R6 are H, or a derivative or analogue thereof, for use in the activation of AMPK.

In some embodiments, an OCH₃ group can cyclize with a neighboring OH group to form a methylene dioxy bridge.

In one embodiment, R1, R2, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R3, R4, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl, or a derivative or analogue thereof, for use in the activation of AMPK.

In some embodiments, an OCH₃ group can cyclize with a neighboring OH group to form a methylene dioxy bridge.

In one embodiment, R1, R2 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3, R4, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2 and R5 are each independently OH; OCH₃; O-glycoside; C-glycoside; acylated O-glycoside; acylated C-glycoside; sulfated O-glycoside; sulfated C-glycoside; a sulfate;

R3, R4, and R6 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2 and R5 are each independently OH; OCH₃; O-glycoside; C-glycoside; acylated O-glycoside; acylated C-glycoside; sulfated O-glycoside; sulfated C-glycoside; a sulfate;

R3, R4, and R6 are H, or a derivative or analogue thereof, for use in the activation of AMPK.

In one embodiment, R1, R2 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate R3, R4, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2 and R5 are each independently OH; OCH₃; O-glycoside; C-glycoside; acylated O-glycoside; acylated C-glycoside; sulfated O-glycoside; sulfated C-glycoside; a sulfate;

R3, R4, and R6 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK.

In some embodiments, an OCH₃ group can cyclize with a neighboring OH group to form a methylene dioxy bridge.

In one embodiment, R1, R2 and R5 are each independently OH; OCH₃; O-glycoside; C-glycoside; acylated O-glycoside; acylated C-glycoside; sulfated O-glycoside; sulfated C-glycoside; a sulfate;

R3, R4, and R6 are H, or a derivative or analogue thereof, for use in the activation of AMPK.

In one preferred embodiment, said compound is compound 2: 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one, a direct activator of AMPK.

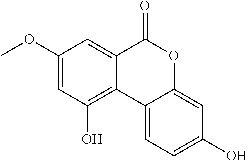 [SMILES: COc1cc(O)c2c(c1)c(=O)oc1cc(O)ccc12]

In one embodiment, the compounds of formula I, II, III, IV and V are obtained from a plant or plant extract.

In another embodiment, the compounds of formula I, II, III, IV and V are obtained by chemical synthesis.

In another embodiment the compound is obtained by the biotransformation of ellagic acids of formula VI, and their ellagitannin precursors

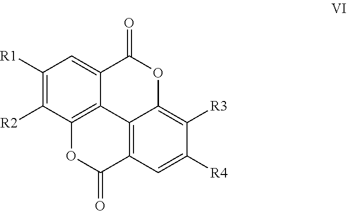

wherein R1, R2, R3 and R4 are each independently H, OH; OCH₃; O-glycoside; sulfated O-glycoside; a sulfate.

In another embodiment the compound is generated in situ in the human body from the metabolism of ellagic acids of formula VI, and their ellagitannin precursors.

The invention also relates to compounds of Formula VII as indirect AMPK activators Formula VII

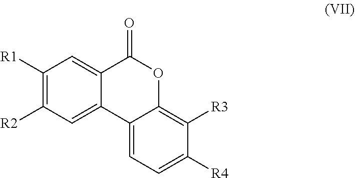

wherein R1, R2, R3, and R4 are each independently OH; OCH₃; O-glycoside; C-glycoside; acylated O-glycoside; acylated C-glycoside; sulfated O-glycoside; sulfated C-glycoside; a sulfate.

In one embodiment, R1, R2, R3, and R4 are each independently OH; OCH₃; O-glycoside; sulfated O-glycoside; a sulfate.

In one embodiment, R1, R2, R3, and R4 are each independently H, OH, OCH₃.

In another embodiment, compounds of Formula VII are selected from the group:

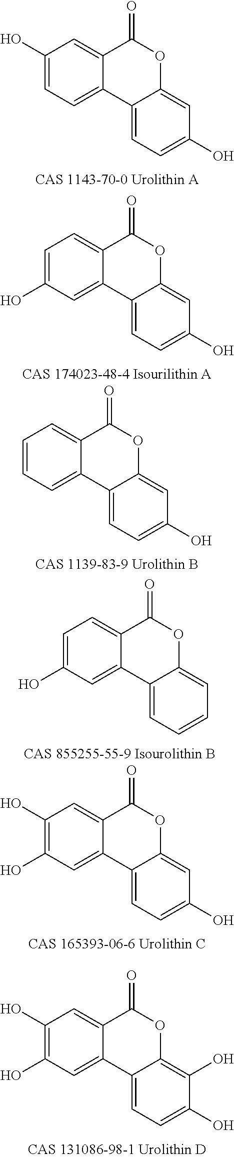

In one preferred embodiment, the indirect AMPK activator of Formula VII is Urolithin B; CAS 1139-83-9.

In another preferred embodiment, the indirect AMPK activator of Formula VII is Urolithin A; CAS 1143-70-0.

In one embodiment, the compounds of formula VII are obtained from a plant or plant extract.

In another embodiment, the compounds of formula VII are obtained by chemical synthesis.

In one embodiment, the combination of the direct AMPK activator and the indirect AMPK activator of the invention, activates of AMPK to treat or prevent a condition, disorder, or disease in a subject.

In one embodiment, the subject is a human or companion animal.

In one embodiment, the subject is a human.

In one embodiment, the subject is an older human.

In one embodiment, the subject is an elderly human.

In one embodiment, the subject is a companion animal.

In one embodiment, the condition, disorder, or disease relates to cardiometabolic health, obesity, type 2 diabetes, non-alcoholic fatty liver disease, cardiovascular disease, and/or cancer.

In one embodiment, the condition, disorder, or disease relates to type 2 diabetes and/or non-alcoholic fatty liver disease.

In one embodiment, the activation of AMPK is a direct activation mechanism from compounds of formulae I, II, III, IV or V wherein these direct AMPK activators act by binding the ADaM binding site at the interface between the alpha-subunit of AMPK and the beta-subunit of AMPK.

In one embodiment, the activation is an indirect activation mechanism from compounds of formula VII wherein these indirect AMPK activators act indirectly to alter the nucleotide status of the cell by lowering ATP in the cell and increasing AMP/ADP, to activate AMPK via the gamma-subunit.

In a preferred embodiment, the activation of AMPK is by the combination of AMPK from a direct activation mechanism from compounds of formulae I, II, III, IV or V wherein these direct AMPK activators act by binding the ADaM binding site at the interface between the alpha-subunit of AMPK and the beta-subunit of AMPK; and an indirect activation mechanism from compounds of formula VII wherein these indirect AMPK activators act indirectly to alter the nucleotide status of the cell by lowering ATP in the cell and increasing AMP/ADP, to activate AMPK via the gamma-subunit.

In one embodiment, the activation of AMPK is in muscle, liver and/or kidney tissues.

In one embodiment, the AMPK comprises an α2 subunit, a β1 subunit, and a γ1 subunit.

In one embodiment, the AMPK comprises an α1 subunit, a β1 subunit, and a γ1 subunit.

In one embodiment, said compound of formula I, II, III, IV, V or VII is obtained from a plant or plant extract.

In one embodiment, said compound of formula I, II, III, IV, V or VII is obtained by chemical synthesis.

In another embodiment the compound is obtained by the biotransformation of ellagic acids of formula VI, and their ellagitannin precursors

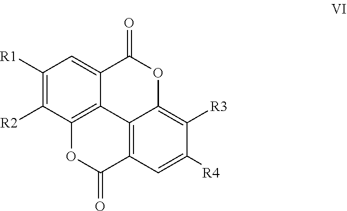

wherein R1, R2, R3 and R4 are each independently H, OH; OCH₃; O-glycoside; sulfated O-glycoside; or a sulfate.

In another embodiment the compound is generated in situ in the human body from the metabolism of ellagic acids of formulae VI, and their ellagitannin precursors.

The present invention also provides a compound of general formula I as described herein for use in the preparation of a medicament for, treating or preventing a condition, disorder, or disease responsive to AMPK activation.

In one embodiment, the compound of general formula I is compound 2 also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one.

In one preferred embodiment, the direct AMPK activator compound of general formula I to V is combined with the indirect AMPK compound of general formula VII to be used in the preparation of a medicament for, treating or preventing a condition, disorder, or disease responsive to AMPK activation.

In one preferred embodiment, the direct AMPK activator compound of general formula I to V is combined with the indirect AMPK compound of general formula VII for use in the preparation of a medicament for treating or preventing type 2 diabetes.

In one preferred embodiment, the direct AMPK activator compound of general formula I to V is combined with the indirect AMPK compound of general formula VII for use in the preparation of a medicament for treating or preventing non-alcoholic fatty liver disease.

The present invention also provides a composition comprising a direct AMPK activator compound of general formula I to V and an indirect AMPK activator of general formula VII formulated specifically as a food, beverage, or dietary supplement.

In one embodiment, the composition is a nutraceutical.

In one preferred embodiment, the food, beverage, dietary supplement or nutraceutical composition comprises a direct AMPK activator compound of general formula I which is compound 2 also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one together with an indirect AMPK activator compound of general formula VII which is compound 1 also known as Urolithin B.

In one preferred embodiment, the food, beverage, dietary supplement or nutraceutical composition comprises a direct AMPK activator compound of general formula I which is compound 2 also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one together with an indirect AMPK activator compound of general formula VII which is Urolithin A.

In one embodiment, the composition combining a direct AMPK activator of formula I to V and an indirect AMPK activator of formula VII further comprises a pharmaceutically acceptable carrier for use in the activation of AMPK.

In one preferred embodiment, the pharmaceutical composition comprises a direct AMPK activator compound of general formula I which is compound 2 also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one and an indirect AMPK activator compound of formula VII which is compound 1 also known as Urolithin B.

In one preferred embodiment, the pharmaceutical composition comprises a direct AMPK activator compound of general formula I which is compound 2 also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one and an indirect AMPK activator compound of formula VII which is Urolithin A.

In one embodiment, the pharmaceutical composition is an oral dosage form.

The present invention also provides a method of administering a therapeutically effective amount of the combination of a direct AMPK compound of general formula I to V and an indirect AMPK compound of general formula VII as described herein for treating or preventing a condition, disorder, or disease responsive to AMPK activation.

In one preferred embodiment, the direct AMPK compound of general formula I is compound also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one and the indirect compound of general formula VII is compound 1 also known as Urolithin B.

In one preferred embodiment, the direct AMPK compound of general formula I is compound also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one and the indirect compound of general formula VII is Urolithin A.

In one embodiment, the disorder responsive to AMPK activation is a metabolic disorder.

In one embodiment, the metabolic disorder is pre-diabetes or diabetes.

In one embodiment, the metabolic disorder of diabetes is accompanied by conditions which may be responsive to AMPK activation, for example, diabetic nephropathy or diabetic neuropathy.

In one embodiment, the metabolic disorder is dyslipidemia.

The present invention also provides a method for activating AMPK in a subject in need thereof, said method comprising administering to the subject in need a composition comprising an effective amount of a direct AMPK activator compound of general formula I to V and an indirect AMPK activator compound of general formula VII as described herein.

In one preferred embodiment, the direct AMPK activator compound of general formula I is compound 2 also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one and the indirect AMPK activator compound of general formula VII is compound 1 also known as Urolithin B.

The present invention also provides an in vitro method of activating AMPK, comprising contacting a direct AMPK activator compound of general formula I to V as described herein, and an indirect AMPK activator compound of general formula VII as described herein, simultaneously or sequentially, with AMPK.

In one embodiment, the in vitro method is cell free.

In one embodiment, the in vitro method is cell based.

In one preferred embodiment, the direct AMPK activator compound of general formula I is compound 2 also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one and the indirect AMPK activator compound of general formula VII is compound 1 also known as Urolithin B.

In another preferred embodiment, the direct AMPK activator compound of general formula I is compound 2 also known as 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one and the indirect AMPK activator compound of general formula VII is Urolithin A.

### DETAILED DESCRIPTION

Combinations of the Invention

The present invention relates to combinations of direct AMPK activators and indirect AMPK activators which are used for activation of AMPK.

The direct AMPK activators of the present invention act by binding the ADaM binding site at the interface between the alpha-subunit of AMPK and the beta-subunit of AMPK. The indirect AMPK activators act by indirectly altering the nucleotide status of the cell by lowering ATP in the cell and increasing AMP/ADP, to activate AMPK via the gamma-subunit.

Compounds of the Invention

A direct AMPK activator compound having the general formula I as described herein has a structure as shown below

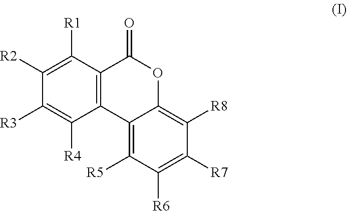

wherein R1, R2, R3, R4, R5, R6, R7, and R8 are each independently H; CH₃; CH₂OH; CHO; COOH; OH; OCH₃; CO—(CH₂)₂—CH₃; O—CO—CH₃; a halogen; O-glycoside; C-glycoside; acylated O-glycoside; acylated C-glycoside; sulfated O-glycoside; sulfated C-glycoside; a sulfate; or a derivative or analogue thereof, for use in the activation of AMPK.

In some embodiments, an OCH₃ group can cyclize with a neighboring OH group to form a methylene dioxy bridge.

In another embodiment, the invention relates to a direct AMPK activator compound of formula (II)

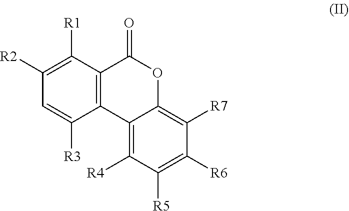

wherein R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4 and R5 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R6, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4, and R5 are each independently OH; OCH₃; O-glycoside; C-glycoside; acylated O-glycoside; acylated C-glycoside; sulfated O-glycoside; sulfated C-glycoside; a sulfate;

R6, and R7 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK. In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4 and R5 are each independently OH; OCH₃; O-glycoside; C-glycoside; acylated O-glycoside; acylated C-glycoside; sulfated O-glycoside; sulfated C-glycoside; a sulfate;

R6 and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R6, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a sulfate;

R6, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R4 and R5 are each independently OH; OCH₃; O-glycoside; C-glycoside; acylated O-glycoside; acylated C-glycoside; sulfated O-glycoside; sulfated C-glycoside; a sulfate;

R6 and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside;

C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside;

acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6 and R7 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6, and R7 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK. In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6, and R7 are each independently OH; OCH₃; O-glycoside; C-glycoside; acylated O-glycoside; acylated C-glycoside; sulfated O-glycoside; sulfated C-glycoside; a sulfate;

R4 and R5 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6 and R7 are each independently OH; OCH₃; O-glycoside;

C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate, or a derivative or analogue thereof, for use in the activation of AMPK. In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a sulfate;

R4 and R5 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside;

acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5 and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5, and R6 are each independently OH; OCH₃; O-glycoside; C-glycoside; acylated O-glycoside; acylated C-glycoside; sulfated O-glycoside; sulfated C-glycoside; a sulfate;

R4, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5, and R6 are each independently OH; OCH₃; O-glycoside; C-glycoside; acylated O-glycoside; acylated C-glycoside; sulfated O-glycoside; sulfated C-glycoside; a sulfate;

R4 and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5 and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate, or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a sulfate;

R4, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a sulfate;

R4 and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, R5, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, R5, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, R5 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are H,

or a derivative or analogue thereof, for use in the activation of AMPK.

In some embodiments, an OCH₃ group can cyclize with a neighboring OH group to form a methylene dioxy bridge.

In another embodiment, the invention relates to a direct AMPK activator compound of formula (III)

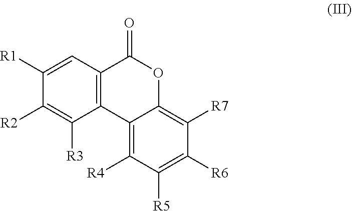

Wherein R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4 and R5 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R6, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4, and R5 are each independently OH; OCH₃; O-glycoside; C-glycoside; acylated O-glycoside; acylated C-glycoside; sulfated O-glycoside; sulfated C-glycoside; a sulfate;

R6, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4 and R5 are each independently OH; OCH₃; O-glycoside; C-glycoside; acylated O-glycoside; acylated C-glycoside; sulfated O-glycoside; sulfated C-glycoside; a sulfate;

R6 and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R6, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R4, and R5 are each independently OH; OCH₃; O-glycoside; C-glycoside; acylated O-glycoside; acylated C-glycoside; sulfated O-glycoside; sulfated C-glycoside; a sulfate;

R6, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R4 and R5 are each independently OH; OCH₃; O-glycoside; C-glycoside; acylated O-glycoside; acylated C-glycoside; sulfated O-glycoside; sulfated C-glycoside; a sulfate;

R6 and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6 and R7 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside;

acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6, and R7 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside;

acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R6, and R7 are each independently OH; OCH₃; O-glycoside; C-glycoside; acylated O-glycoside; acylated C-glycoside; sulfated O-glycoside; sulfated C-glycoside; a sulfate;

R4 and R5 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6 and R7 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R6, and R7 are each independently OH; OCH₃; O-glycoside; C-glycoside; acylated O-glycoside; acylated C-glycoside; sulfated O-glycoside; sulfated C-glycoside; a sulfate;

R4 and R5 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5 and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5, and R6 are each independently OH; OCH₃; O-glycoside; C-glycoside; acylated O-glycoside; acylated C-glycoside; sulfated O-glycoside; sulfated C-glycoside; a sulfate;

R4, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside;

acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R5, and R6 are each independently OH; OCH₃; O-glycoside; C-glycoside; acylated O-glycoside; acylated C-glycoside; sulfated O-glycoside; sulfated C-glycoside; a sulfate;

R4 and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5 and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside;

C-glycoside; acylated O-glycoside; acylated C-glycoside; sulfated O-glycoside; sulfated C-glycoside; a sulfate;

R4, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, R5, and R6 are each independently OH; OCH₃; O-glycoside; C-glycoside; acylated O-glycoside; acylated C-glycoside; sulfated O-glycoside; sulfated C-glycoside; a sulfate;

R4 and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, R5, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, R5, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R2 are each independently OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, R5 and R7 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R6 are each independently OH; OCH₃; O-glycoside; C-glycoside; acylated O-glycoside; acylated C-glycoside; sulfated O-glycoside; sulfated C-glycoside; a sulfate;

R4, R5, and R7 are H,

or a derivative or analogue thereof, for use in the activation of AMPK.

In some embodiments, an OCH₃ group can cyclize with a neighboring OH group to form a methylene dioxy bridge.

In another embodiment, the invention relates to a direct AMPK activator compound of formula (IV)

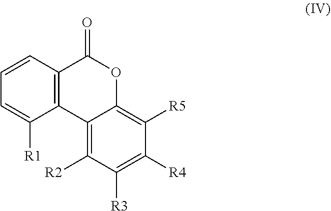

wherein R1, R2, and R3 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R4, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, and R3 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1, R2 and R3 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R4, and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, and R3 are each independently OH; OCH₃; O-glycoside; C-glycoside; acylated O-glycoside; acylated C-glycoside; sulfated O-glycoside; sulfated C-glycoside; a sulfate;

R4, and R5 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2 and R3 are each independently OH; OCH₃; O-glycoside; C-glycoside; acylated O-glycoside; acylated C-glycoside; sulfated O-glycoside; sulfated C-glycoside; a sulfate;

R4 and R5 are H, or a derivative or analogue thereof, for use in the activation of AMPK.

In some embodiments, an OCH₃ group can cyclize with a neighboring OH group to form a methylene dioxy bridge.

In one embodiment, R1, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R2, and R3 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R4 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, and R3 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1, R4 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R2, and R3 are H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R4, and R5 are each independently OH; OCH₃; O-glycoside; C-glycoside; acylated O-glycoside; acylated C-glycoside; sulfated O-glycoside; sulfated C-glycoside; a sulfate;

R2, and R3 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R4, and R5 are each independently OH; OCH₃; O-glycoside; C-glycoside; acylated O-glycoside; acylated C-glycoside; sulfated O-glycoside; sulfated C-glycoside; a sulfate;

R2 and R3 are H,

or a derivative or analogue thereof, for use in the activation of AMPK.

In some embodiments, an OCH₃ group can cyclize with a neighboring OH group to form a methylene dioxy bridge.

In one embodiment, R1, R3, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R2, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R3, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1, R3 and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R2, and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R3, and R4 are each independently OH; OCH₃; O-glycoside; C-glycoside; acylated O-glycoside; acylated C-glycoside; sulfated O-glycoside; sulfated C-glycoside; a sulfate;

R2, and R5 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R3, and R4 are each independently OH; OCH₃; O-glycoside; C-glycoside; acylated O-glycoside; acylated C-glycoside; sulfated O-glycoside; sulfated C-glycoside; a sulfate;

R2 and R5 are H,

or a derivative or analogue thereof, for use in the activation of AMPK.

In some embodiments, an OCH₃ group can cyclize with a neighboring OH group to form a methylene dioxy bridge.

In one embodiment, R1, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R2, R3, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R3, and R5 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R2, R3, and R5 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R4 are each independently OH; OCH₃; O-glycoside; C-glycoside; acylated O-glycoside; acylated C-glycoside; sulfated O-glycoside; sulfated C-glycoside; a sulfate;

R2, R3, and R5 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 and R4 are each independently OH; OCH₃; O-glycoside; C-glycoside; acylated O-glycoside; acylated C-glycoside; sulfated O-glycoside; sulfated C-glycoside; a sulfate;

R2, R3, and R5 are H,

or a derivative or analogue thereof, for use in the activation of AMPK.

In another embodiment, the invention relates to a direct AMPK activator compound of formula (V)

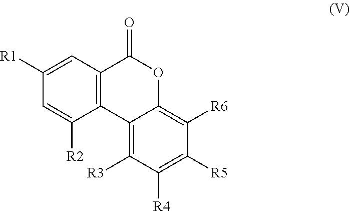

wherein R1, R2, R3, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R5 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R5 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R3 and R4 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R5, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R3, and R4 are each independently OH; OCH₃; O-glycoside; C-glycoside; acylated O-glycoside; acylated C-glycoside; sulfated O-glycoside; sulfated C-glycoside; a sulfate;

R5, and R6 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R3 and R4 are each independently OH; OCH₃; O-glycoside; C-glycoside; acylated O-glycoside; acylated C-glycoside; sulfated O-glycoside; sulfated C-glycoside; a sulfate;

R5 and R6 are H,

or a derivative or analogue thereof, for use in the activation of AMPK.

In some embodiments, an OCH₃ group can cyclize with a neighboring OH group to form a methylene dioxy bridge.

In one embodiment, R1, R2, R3 and R4 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R5, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3, and R4 are each independently OH; OCH₃; O-glycoside; C-glycoside; acylated O-glycoside; acylated C-glycoside; sulfated O-glycoside; sulfated C-glycoside; a sulfate;

R5, and R6 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R3 and R4 are each independently OH; OCH₃; O-glycoside; C-glycoside; acylated O-glycoside; acylated C-glycoside; sulfated O-glycoside; sulfated C-glycoside; a sulfate;

R5 and R6 are H,

or a derivative or analogue thereof, for use in the activation of AMPK.

In some embodiments, an OCH₃ group can cyclize with a neighboring OH group to form a methylene dioxy bridge.

In one embodiment, R1, R2, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

R3 and R4 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R5, and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R4 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R5 and R6 are each independently OH; OCH₃; O-glycoside; C-glycoside; acylated 0-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3 and R4 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R5, and R6 are each independently OH; OCH₃; O-glycoside; C-glycoside; acylated O-glycoside; acylated C-glycoside; sulfated O-glycoside; sulfated C-glycoside; a sulfate;

R3, and R4 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R5, and R6 are each independently OH; OCH₃; O-glycoside; C-glycoside; acylated O-glycoside; acylated C-glycoside; sulfated O-glycoside; sulfated C-glycoside; a sulfate;

R3 and R4 are H,

or a derivative or analogue thereof, for use in the activation of AMPK.

In some embodiments, an OCH₃ group can cyclize with a neighboring OH group to form a methylene dioxy bridge.

In one embodiment, R1, R2, R5 and R6 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3, and R4 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R5, and R6 are each independently OH; OCH₃; O-glycoside; C-glycoside; acylated O-glycoside; acylated C-glycoside; sulfated O-glycoside; sulfated C-glycoside; a sulfate;

R3, and R4 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R5, and R6 are each independently OH; OCH₃; O-glycoside; C-glycoside; acylated O-glycoside; acylated C-glycoside; sulfated O-glycoside; sulfated C-glycoside; a sulfate;

R3 and R4 are H,

or a derivative or analogue thereof, for use in the activation of AMPK.

In some embodiments, an OCH₃ group can cyclize with a neighboring OH group to form a methylene dioxy bridge.

In one embodiment, R1, R2, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R3 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2, R4, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R4 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R4, and R5 are each independently OH; OCH₃; O-glycoside; C-glycoside; acylated O-glycoside; acylated C-glycoside; sulfated O-glycoside; sulfated C-glycoside; a sulfate;

R3, and R6 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside;

acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2, R4, and R5 are each independently OH; OCH₃; O-glycoside; C-glycoside; acylated O-glycoside; acylated C-glycoside; sulfated O-glycoside; sulfated C-glycoside; a sulfate;

R3 and R6 are H,

or a derivative or analogue thereof, for use in the activation of AMPK.

In some embodiments, an OCH₃ group can cyclize with a neighboring OH group to form a methylene dioxy bridge.

In one embodiment, R1, R2, R4 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R4, and R5 are each independently OH; OCH₃; O-glycoside; C-glycoside; acylated O-glycoside; acylated C-glycoside; sulfated O-glycoside; sulfated C-glycoside; a sulfate;

R3, and R6 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2, R4, and R5 are each independently OH; OCH₃; O-glycoside; C-glycoside; acylated O-glycoside; acylated C-glycoside; sulfated O-glycoside; sulfated C-glycoside; a sulfate;

R3 and R6 are H,

or a derivative or analogue thereof,

or a derivative or analogue thereof, for use in the activation of AMPK.

In some embodiments, an OCH₃ group can cyclize with a neighboring OH group to form a methylene dioxy bridge.

In one embodiment, R1, R2, and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl;

R3, R4, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl,

or a derivative or analogue thereof, for use in the activation of AMPK.

In some embodiments, an OCH₃ group can cyclize with a neighboring OH group to form a methylene dioxy bridge.

In one embodiment, R1, R2 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R3, R4, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl, or a derivative or analogue thereof,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate;

R3, R4, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside;

acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2 and R5 are each independently OH; OCH₃; O-glycoside; C-glycoside; acylated O-glycoside; acylated C-glycoside; sulfated O-glycoside; sulfated C-glycoside; a sulfate;

R3, R4, and R6 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1 is OH; OCH₃; O-glycoside; C-glycoside; acylated O-glycoside;

acylated C-glycoside; sulfated O-glycoside; sulfated C-glycoside; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl;

R2 and R5 are each independently OH; OCH₃; O-glycoside; C-glycoside; acylated O-glycoside; acylated C-glycoside; sulfated O-glycoside; sulfated C-glycoside; a sulfate;

R3, R4, and R6 are H,

or a derivative or analogue thereof, for use in the activation of AMPK.

In one embodiment, R1, R2 and R5 are each independently OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate R3, R4, and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2 and R5 are each independently OH; OCH₃; O-glycoside; C-glycoside; acylated O-glycoside; acylated C-glycoside; sulfated O-glycoside; sulfated C-glycoside; a sulfate;

R3, R4, and R6 are each independently H; OH; OCH₃; O-glycoside; C-glycoside; acylated O-glycoside; acylated C-glycoside; sulfated O-glycoside; sulfated C-glycoside; a sulfate,

or a derivative or analogue thereof, for use in the activation of AMPK.

In some embodiments, an OCH₃ group can cyclize with a neighboring OH group to form a methylene dioxy bridge.

In one embodiment, R1, R2 and R5 are each independently OH; OCH₃; O-glycoside; C-glycoside; acylated O-glycoside; acylated C-glycoside; sulfated O-glycoside; sulfated C-glycoside; a sulfate;

R3, R4, and R6 are H,

or a derivative or analogue thereof, for use in the activation of AMPK.

In one preferred embodiment, said compound is compound 2: 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one.

The invention also relates to compounds of Formula VII as indirect AMPK activators wherein Formula VII

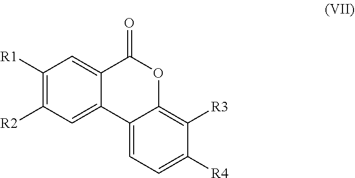

wherein R1, R2, R3, and R4 are each independently OH; OCH₃; O-glycoside; C-glycoside; acylated O-glycoside; acylated C-glycoside; sulfated O-glycoside; sulfated C-glycoside; a sulfate.

In one embodiment R1, R2, R3, and R4 are each independently OH; OCH₃; O-glycoside; sulfated O-glycoside; a sulfate.

In one embodiment, R1, R2, R3, and R4 are each independently H, OH, OCH₃.

In another embodiment, compounds of Formula VII are selected from the group:

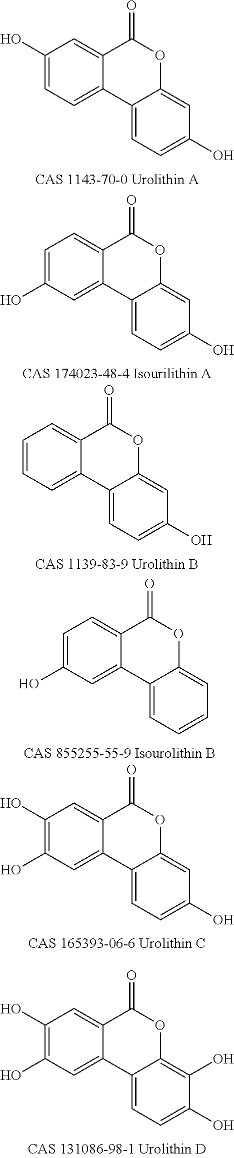

In one preferred embodiment, the indirect AMPK activator of Formula VII is Urolithin B; CAS 1139-83-9.

In another preferred embodiment, the indirect AMPK activator of Formula VII is Urolithin A; CAS 1143-70-0.

In one embodiment, the compounds of formulae I, II, III, IV, V, and VII are obtained from a plant or plant extract.

In another embodiment, the compounds of formulae I, II, III, IV, V, and VII are obtained by chemical synthesis.

In another embodiment the compound is obtained by the biotransformation of ellagic acids of formula VI, and their ellagitannin precursors.

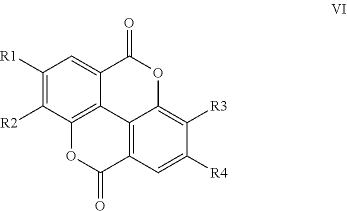

wherein R1, R2, R3 and R4 are each independently H, OH; OCH₃; O-glycoside; sulfated O-glycoside; a sulfate

In another embodiment the compound is generated in situ in the human body from the metabolism of ellagic acids of formula VI, and their ellagitannin precursors.

### Definitions

AMPK Activator Compounds

Direct activators bind directly to AMPK at the alpha, beta and/or gamma subunit.

The preferred direct AMPK activators act by binding the ADaM binding site at the interface between the alpha-subunit of AMPK and the beta-subunit of AMPK.

The indirect AMPK activators act by indirectly altering the nucleotide status of the cell by lowering ATP in the cell and increasing AMP/ADP, to activate AMPK via the gamma-subunit.

General Chemistry Terminology

The term “alkyl” refers to a branched or unbranched saturated hydrocarbon chain having from 1 to 20 carbon atoms, or from 1 to 15 carbon atoms, or from 1 to 10 carbon atoms, or from 1 to 7 carbon atoms, or from 1 to 5 carbon atoms, or from 1 to 3 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:

1) an alkyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)— cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or 2) an alkyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or
3) an alkyl chain as defined above that has both 1, 2, 3, 4 or 5 substituents as defined above and is also interrupted by 1-5 atoms (e.g. 1, 2, 3, 4 or 5 atoms) as defined above.

4) an alkyl chain as defined above in which one of the methylene group is replaced by a carbonyl group to give an oxo group. Non limiting examples include —CH₂—CH₂—CO—CH₂—CH₃, —CH₂—CO—(CH₂)n-CH₃ in which n=2, 4, or 6.

5) an alkyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

The term “alkenyl” refers to a type of alkyl chain in which two atoms of the alkyl chain form a double bond that is not part of an aromatic group. That is, an alkenyl chain contains the pattern R—C(R)═C(R)—R, wherein R refers to the remaining portions of the alkenyl chain, which may be the same or different. Non-limiting examples of an alkenyl chain include —C(CH₃)═CH—CH₃, —CH═CH₂, —C(CH₃)═CH₂, —CH═CH—CH₃, —C(CH₃)═CH—CH₃, —CH₂—CH═C(CH₃)₂, and —C(CH₃)₂—CH═CH₂. The alkenyl moiety may be branched, straight chain, or cyclic (in which case, it would also be known as a “cycloalkenyl” group). Alkenyl chains can be optionally substituted.

The alkenyl chain as defined above can be interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2.

The alkenyl chain as defined above can be interrupted by an oxo group.

One of the methylene of the alkenyl chain as defined above can be replaced by an oxo group, and the chain can either have 1, 2, 3, 4 or 5 substituents as defined above, or be interrupted by 1-5 atoms (e.g. 1, 2, 3, 4 or 5 atoms) as defined above, or can have both 1, 2, 3, 4 or 5 substituents as defined above and be also interrupted by 1-5 atoms (e.g. 1, 2, 3, 4 or 5 atoms) as defined above.

The term “alkynyl” refers to a type of alkyl chain in which two atoms of the alkyl chain form a triple bond. That is, an alkynyl chain contains the pattern R—C≡C—R, wherein R refers to the remaining portions of the alkynyl chain, which may be the same or different. Non-limiting examples of an alkynyl chain include —C≡CH, —C≡C—CH₃ and —C≡C—CH₂—CH₃. The “R” portion of the alkynyl moiety may be branched, straight chain, or cyclic. Alkynyl chains can be optionally substituted.

The alkynyl chain as defined above can be interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2

The alkynyl chain as defined above can be interrupted by an oxo group.

One of the methylene of the alkynyl chain as defined above can be replaced by an oxo group, and the chain can either have 1, 2, 3, 4 or 5 substituents as defined above, or be interrupted by 1-5 atoms (e.g. 1, 2, 3, 4 or 5 atoms) as defined above, or can have both 1, 2, 3, 4 or 5 substituents as defined above and be also interrupted by 1-5 atoms (e.g. 1, 2, 3, 4 or 5 atoms) as defined above.

The term “polyunsaturated” refers to

1) A chain known as polyalkenyl in which more than one pair of atoms of the alkyl chain form a double bond that is not part of an aromatic group. That is, a polyalkenyl chain contains from 2 to 8 R—C(R)═C(R)—R patterns, wherein R refers to the remaining portions of the alkenyl chain, which may be the same or different. The polyalkenyl moiety may be branched, or straight chain. Non-limiting examples of a polyalkenyl chain include —CH═CH—CH═CH—CH₃, —(CH₂)₂—CH═CH—CH═CH—(CH₂)₂—CH₃, —CH₂—CH═C(CH₃)—CH₂—CH₂—CH═C(CH₃)₂, and —CH₂—CH═C(CH₃)—CH₂—CH₂—CH═C(CH₃)—CH₂—CH₂—CH═C(CH₃)₂. The polyalkenyl moiety containing two double bonds may be cyclic (in which case, it would also be known as a “cyclodialkenyl” group). Non limiting example of cyclodialkenyl groups include cyclopentadiene and cyclohexadiene groups. Polyalkenyl chains can be optionally substituted.

2) A chain known as polyalkynyl in which more than one pair of atoms of the alkyl chain form a triple bond. That is, a polyalkynyl chain contains from 2 to 8 R—C≡C—R patterns, wherein R refers to the remaining portions of the alkynyl chain, which may be the same or different. Non-limiting example of a polyalkynyl chain include —CH₂—CH₂—C≡C—C≡CH. The “R” portion of the polyalkynyl moiety may be branched, straight chain, or cyclic. Alkynyl chains can be optionally substituted.

3) A type of alkyl chain in which at least one pair of atoms of the alkyl chain form a double bond and one pair of atoms of the alkyl chain form a triple bond. That is, a polyunsaturated chain contains both R—C(R)═C(R)—R and R—C≡C—R patterns, wherein R refers to the remaining portions of the polyunsaturated chain, which may be the same or different and the total number of unsaturated bonds may vary from 2 to 8. Non-limiting examples this type of polyunsaturated chain include —CH₂—CH═CH—C≡CH. The “R” portion of the polyunsaturated moiety may be branched, straight chain, or cyclic. Polyunsaturated chains can be optionally substituted.

4) A polyunsaturated chain as defined above in paragraphs 1-3, that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl.

5) A polyunsaturated chain as defined above in paragraphs 1-3, in which one of the methylene group is replaced by a carbonyl group to give an oxo group.

6) A polyunsaturated chain as defined above in paragraphs 1-3, in which one of the methylene group is replaced by a carbonyl group to give an oxo group, and is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can form part of a ring system. As used herein, the term “ring system” refers to two or more rings, wherein two or more of the rings are fused. The term “fused” refers to structures in which two or more rings share one or more bonds.

The term “halogen” may refer to a fluorine atom, a chlorine atom, a bromine atom or an iodine atom.

The term “glycoside” refers to a compound in which at least one sugar is bound to another functional group via a glycosidic bond. Typically the glycosidic chain can comprise 1 to 4 sugar units.

The term “glycosidic bond” refers to a bond formed between the hemiacetal or hemiketal group of a sugar and the chemical group of a compound. The chemical group can be —OH (O-glycoside), or —CR1R2R3 (C-glycoside).

The terms “acylated O-glycoside” and “acylated C-glycoside” refer to a compound in which at least one hydroxyl of the glycosidic chain is esterified by an organic acid. Typical examples or organic acid may comprise acetic, substituted benzoic, cinnamic (caffeic, ferulic, p-coumaric), and/or phenylpropanoic (dihydrocaffeic) acids.

The terms “sulfated O-glycoside” and “sulfated C-glycoside” refer to a compound in which at least one hydroxyl of the glycosidic chain is esterified by sulfuric acid.

The term “methylene dioxy” may refer to functional group with the structural formula R—O—CH₂—O—R′, connected to the rest of a molecule by two chemical bonds.

The term “analogue” as used herein is understood to refer to a compound having a structure similar to that of another one, but differing from it in respect of a certain component. A “derivative” is a compound that can be imagined to arise or is actually be synthesized from a parent compound by replacement of one or more atoms with another atom or group of atoms.

Compound or Composition Thereof

It is understood that according to certain embodiments, the compound of the invention or composition thereof may be a nutraceutical composition, pharmaceutical composition, functional food, functional nutrition product, medical food, medical nutrition product, or a dietary supplement.

The terms “nutraceutical” combines the words “nutrition” and “pharmaceutical”. It is a food or food product that provides health and medical benefits, including the prevention and treatment of a condition, disorder, or disease. A nutraceutical is a product isolated or purified from foods that is generally sold in medicinal forms not usually associated with food. A nutraceutical is demonstrated to have a physiological benefit or provide protection against a condition, disorder, or disease. Such products may range from isolated nutrients, dietary supplements and specific diets to genetically engineered foods, herbal products, and processed foods such as cereals, soups, and beverages.

The term “nutraceutical” as used herein denotes usefulness in both nutritional and pharmaceutical fields of application. Thus, novel nutraceutical compositions can be used as supplements to food and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellifying agents, gel-forming agents, antioxidants and antimicrobials.

Moreover, a multi-vitamin and mineral supplement may be added to nutraceutical compositions of the invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions of the invention may be in any galenic form that is suitable for administering to the body, especially in any form that is conventional for oral administration, e.g. in solid forms such as food or feed, food or feed premix, fortified food or feed, tablets, pills, granules, dragees, capsules and effervescent formulations such as powders and tablets, or in liquid forms, such as solutions, emulsions or suspensions as e.g. beverages, pastes and oily suspensions. The pastes may be incorporated in hard or soft shell capsules, whereby the capsules feature e.g. a matrix of (fish, swine, poultry, cow) gelatine, plant proteins or lignin sulfonate. Examples for other application forms are those for transdermal, parenteral or injectable administration. The dietary and pharmaceutical compositions may be in the form of controlled (delayed) release formulations.

Beverages encompass non-alcoholic and alcoholic drinks as well as liquid preparations to be added to drinking water and liquid food. Non-alcoholic drinks are e.g. soft drinks, sports drinks, fruit juices, teas and milk-based drinks. Liquid foods are e.g. soups and dairy products. The nutraceutical composition comprising the compound of the invention may be added to a soft drink, an energy bar, or a candy.

If the nutraceutical composition is a pharmaceutical formulation and the composition further contains pharmaceutically acceptable excipients, diluents or adjuvants then standard techniques may be used for their formulation, as e.g. disclosed in Remington's Pharmaceutical Sciences, 20th edition Williams & Wilkins, PA, USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives.

“Functional food”, “functional nutrition product”, “medical food” and “medical nutrition product” relate to any healthy food claimed to have a health-promoting or disease-preventing property beyond the basic function of supplying nutrients. The general category of functional foods includes processed food or foods fortified with health-promoting additives, like “vitamin-enriched” products.

The terms “food,” “food product” and “food composition” or “diet product” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet.

A dietary supplement, also known as food supplement or nutritional supplement, is a preparation intended to supplement the diet and provide nutrients, such as vitamins, minerals, fibre, fatty acids, or amino acids that may be missing or may not be consumed in sufficient quantities in a person's diet. Some countries define dietary supplements as foods, while in others they are defined as drugs or natural health products. Supplements containing vitamins or dietary minerals are included as a category of food in the Codex Alimentarius, a collection of internationally recognized standards, codes of practice, guidelines and other recommendations relating to foods, food production and food safety. These texts are drawn up by the Codex Alimentarius Commission, an organization that is sponsored by the Food and Agriculture Organization of the United Nations (FAO) and the World Health Organization (WHO).

Compositions intended for an animal, include food compositions to supply the necessary dietary requirements for an animal, animal treats (e.g., biscuits), and/or dietary supplements.

The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of the UFA and NORC, and

B vitamins and antioxidants. This permits the supplement to be administered to the animal in small amounts, or in the alternative, can be diluted before administration to an animal. The dietary supplement may require admixing, or can be admixed with water or other diluent prior to administration to the animal.

“Pet food” or “pet treat compositions” comprise from about 15% to about 50% crude protein.

The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof. The compositions may further comprise from about 5% to about 40% fat. The compositions may further comprise a source of carbohydrate. The compositions may comprise from about 15% to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

In some embodiments, the ash content of the pet food composition ranges from less than 1% to about 15%, and in one aspect, from about 5% to about 10%.

The moisture content can vary depending on the nature of the pet food composition. In a one embodiment, the composition can be a complete and nutritionally balanced pet food. In this embodiment, the pet food may be a “wet food”, “dry food”, or food of intermediate moisture content. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15% or 20%, and therefore is presented, for example, as small biscuit-like kibbles. In one embodiment, the compositions have moisture content from about 5% to about 20%. Dry food products include a variety of foods of various moisture contents, such that they are relatively shelf-stable and resistant to microbial or fungal deterioration or contamination. Also included are dry food compositions which are extruded food products, such as pet foods, or snack foods for companion animals.

Methods of Administration of Compound or Composition Thereof

The compound of the invention or composition thereof is preferably administered by oral administration. In some embodiments, the compound of the invention or composition thereof may be administered by intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

The composition of the invention can have an acute effect that can be seen in less than one month. Additionally or alternatively, the composition can have a long term effect, and thus various embodiments comprise administration of the composition to the individual (e.g., orally) for a time period of at least one month; preferably at least two months, more preferably at least three, four, five or six months; most preferably for at least one year. During the time period, the composition can be administered to the individual at least one day per week; preferably at least two days per week, more preferably at least three, four, five or six days per week; most preferably seven days per week. The composition can be administered in a single dose per day or in multiple separate doses per day. In one embodiment, a single dose is not less than about 100 mg. In one embodiment, a single dose is not more than about 1000 mg. In one embodiment, a single dose is between about 100 mg and about 1000 mg.

AMPK Activation Terminology

As used herein, an “AMPK activator” refers to a compound that either increases the phosphorylation of downstream substrates of (phosphorylated or not) AMPK, and/or that increases the phosphorylation of AMPK.

As used herein, a “direct AMPK activator” refers to a compound that activates AMPK via direct interaction with at least one of its subunits. The direct AMPK activators act by binding the ADaM binding site at the interface between the alpha-subunit of AMPK and the beta-subunit of AMPK.

As used herein, an “indirect AMPK activator” refers to a compound that activates AMPK in an indirect manner, namely, they do not bind directly to AMPK but alter the nucleotide status of the cell by lowering ATP in the cell and increasing AMP/ADP, to activate AMPK via the gamma-subunit.

In one preferred embodiment, the combination activates AMPKα2β1γ1. As used herein, a condition, disorder, or disease “responsive to AMPK activation” refers to one in which the symptoms would be alleviated, or the course of which would be beneficially modified, through activation of AMPK, including without limitation, a metabolic disorder, diabetes, dyslipidemia, hypertension, being overweight, and obesity. For example, the metabolic disorder of diabetes is accompanied by conditions such as diabetic nephropathy or diabetic neuropathy which may be responsive to AMPK activation.

Medical Terminology

As used herein, the term “diabetes” includes insulin-dependent diabetes mellitus (i.e. IDDM, also known as type 1 diabetes) non-insulin-dependent diabetes mellitus (i.e. NIDDM, also known as type 2 diabetes), and prediabetes. Type 1 diabetes is the result of an absolute deficiency of insulin, the hormone which regulates glucose utilization. Type 2 diabetes often occurs in the face of normal, or even elevated levels of insulin and appears to be the result of the inability of tissues to respond appropriately to insulin. This is termed “insulin resistance”. Most type 2 diabetic patients are also overweight or obese. One of the criteria for diagnosing diabetes is the fasting plasma glucose level. A diabetic subject has a fasting plasma glucose level of greater than or equal to 126 mg/dl. A prediabetic subject is someone suffering from prediabetes. A prediabetic subject is a subject with impaired fasting glucose (a fasting plasma glucose level of greater than or equal to 100 mg/dl and less than 126 mg/dl); or impaired glucose tolerance (a 2-hour plasma glucose level of >140 mg/dl and <200 mg/dl); or insulin resistance, resulting in an increased risk of developing diabetes. Prevention of type 2 diabetes includes treatment of prediabetes.

As used herein, the term “dyslipidemia” encompasses abnormal levels of any lipid fractions as well as specific lipoprotein abnormalities. For example, it refers to elevation of plasma cholesterol and/or elevation of triglycerides and/or elevation of free fatty acids and/or low high-density lipoprotein (HDL) level and/or high low-density lipoprotein (LDL) level and/or high very low-density lipoprotein (VLDL) level. Dyslipidemia may for example contribute to the development of atherosclerosis and ultimately symptomatic vascular disease including coronary heart disease. Dyslipidemia may or may not be associated with diabetes.

As used herein, the term “metabolic disorder” encompasses any abnormal chemical and enzymatic reactions disrupting normal metabolism due to environmental and genetic factors (environmental factors include physical activity, nutrition), leading to excessive levels or deficiency of certain substances and dysfunction of energy homeostasis. Non-limiting examples of metabolic disorders include diabetes, dyslipidemia, hypertension, being overweight, obesity, and any combination thereof.

As used herein, “AMPK-related diseases” includes pathologic or pathogenomic conditions in which the activation of AMPK provides a salutary effect. Examples of such diseases or conditions include obesity, diabetes, metabolic syndrome, acute inflammatory lung injury, heart disease, reperfusion ischemia, cancer, aging, retinal degeneration, cardiac hypertrophy, non-alcoholic fatty liver disease, hypertension, albuminuria, sporadic Alzheimer's disease, muscular dystrophy, and osteoarthritis. In addition, “AMPK-related conditions” include conditions where the activation of AMPK improves the condition associated with the primary “AMPK-related disease”. For example, diabetic nephropathy (Salotto et al. (2017) J. Pharma and Expt Thera. 361:303-311) or diabetic neuropathy are “AMPK-related conditions” which may be associated with the “AMPK-related disease” of diabetes.

“Prevention” or “preventing” includes reduction of risk and/or severity of a condition, disorder, or disease.

The terms “treatment,” “treating,”, “treat”, “attenuate” and “alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder, and include treatment of patients at risk of contracting a disease or suspected to have contracted a disease, as well as patients who are ill or have been diagnosed as suffering from a disease or medical condition. The term does not necessarily imply that a subject is treated until total recovery. These terms also refer to the maintenance and/or promotion of health in a subject not suffering from a disease but who may be susceptible to the development of an unhealthy condition. These terms are also intended to include the potentiation or otherwise enhancement of one or more primary prophylactic or therapeutic measure. The terms “treatment,” “treat,” “attenuate” and “alleviate” are further intended to include the dietary management of a disease or condition or the dietary management for prophylaxis or prevention a disease or condition. A treatment can be patient- or doctor-related.

Obesity, which is an excess of body fat relative to lean body mass, is a chronic disease that is highly prevalent in modern society. It is associated not only with a social stigma, but also with decreased life span and numerous medical problems, including adverse psychological development, coronary artery disease, hypertension, stroke, diabetes, hyperlipidemia, and some cancers, (see, e.g., Nishina, et al., Metab. 43:554-558, 1994; Grundy and Barnett, Dis. Mon. 36:641-731, 1990; Rissanen, et al., British Medical Journal, 301:835-837, 1990).

“Obesity related disorders” refers to those diseases or conditions where excessive body weight or high “body mass index (BMI)” has been implicated in the progression or suppression of the disease or condition. Representative examples of obesity related disorders include, without limitation diabetes, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia. See, Harrison's Principles of Internal Medicine, 13th Ed., McGraw Hill Companies Inc., New York (1994). Examples, without limitation, of inflammatory conditions include diseases of the digestive organs (such as ulcerative colitis, Crohn's disease, pancreatitis, gastritis, benign tumor of the digestive organs, digestive polyps, hereditary polyposis syndrome, colon cancer, rectal cancer, stomach cancer and ulcerous diseases of the digestive organs), stenocardia, myocardial infarction, sequelae of stenocardia or myocardial infarction, senile dementia, cerebrovascular dementia, immunological diseases and cancer in general.

The term “subject” or “individual” means any animal, including a human, that could benefit from one or more of the compounds, compositions or methods disclosed herein. Generally, the subject is a human or an avian, bovine, canine, equine, feline, hircine, lupine, murine, ovine or porcine animal. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Preferably, the subject is a human or a companion animal such as a dog or cat. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly subjects. For other animals, an “older adult” has exceeded 50% of the average lifespan for its particular species and/or breed within a species. An animal is considered “elderly” if it has surpassed 66% of the average expected lifespan, preferably if it has surpassed the 75% of the average expected lifespan, more preferably if it has surpassed 80% of the average expected lifespan. An elderly cat or dog has an age from birth of at least about 7 years.

As used herein, an “effective amount” is an amount that prevents a deficiency, treats a disorder, condition, or disease in a subject or, more generally, reduces symptoms, manages progression of the diseases or provides a nutritional, physiological, or medical benefit to the subject. The relative terms “improved,” “increased,” “enhanced” and the like refer to the effects of the composition disclosed herein relative to a composition lacking one or more ingredients and/or having a different amount of one or more ingredients, but otherwise identical.

### General Terminology

As used herein, the singular forms “a,” “an” and “the” include plural referents unless the context clearly dictates otherwise. Thus, for example, reference to “a component” or “the component” includes two or more components.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 10th Ed., McGraw Hill Companies Inc., New York (2001). Standard medical terminology used herein has the meaning defined in Stedman's Medical Dictionary, 27th Edition, with veterinary medicine insert.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or compounds, but may also include additional features or compounds. The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the claims. Numerous specific details are set forth in the description in order to provide a thorough understanding of the present invention. The present invention may be practiced without some or all of these specific details. In other instances, well known methods and protocols have not been described in detail, in order not to unnecessarily obscure the present invention.

### BRIEF DESCRIPTION OF FIGURES

FIG. 1. Compound 1 increases the phosphorylation of the AMPK substrate, acetyl-CoA carboxylase (ACC), in U-2 OS Flp-In T-REx mammalian cells.

U-2 OS cells were treated with varying concentrations of compound 1 for 30 mins at 37 C. Phosphorylation of ACC was assessed using the HTRF Cisbio (pACC kit). Results are displayed as the ratio 665/620 nm (±SEM) of 3 independent experiments.

Compound 1: indirect activator (Urolithin B) 3-hydroxy-6H-benzo[c]chromen-6-one; CAS: 1139-83-9

FIG. 2. Compound 2 improves the dose-response curve of Compound 1, for activation of AMPK in cells.

U-2 OS cells were treated with varying concentrations of compound 1 for 30 mins at 37 C in the presence or absence of 11 μM of compound 2. Phosphorylation of ACC was assessed using the HTRF Cisbio (pACC kit). Results are displayed as the ratio 665/620 nm (±SEM) of 3 independent experiments.

Compound 1: indirect activator (Urolithin B) 3-hydroxy-6H-benzo[c]chromen-6-one; CAS: 1139-83-9

Compound 2: direct activator (Benzocoumarin) 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one

FIG. 3. Compound 2 does not improve the dose-response curve of Compound 1 in cells stably expressing an ADaM-binding site AMPK mutant (β1 S108A).

The β1 WT or β1 S108A mutant were stably expressed in AMPKβ1β2 double knockout cells and were treated with varying concentrations of compound 1 for 30 mins at 37 C in the presence or absence of 11 μM of compound 2. Phosphorylation of ACC was assessed using the HTRF Cisbio (pACC kit). Results are displayed as the average fold increase in activation (±SEM) of 3 independent experiments.

Compound 1: indirect activator (Urolithin B) 3-hydroxy-6H-benzo[c]chromen-6-one; CAS: 1139-83-9

Compound 2: direct activator (Benzocoumarin) 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one

FIG. 4. Compound 2 does not improve the dose-response curve of compound 1 in cells stably expressing the β2 isoform.

U-2 OS cells AMPKβ1β2 double knockout cells stably expressing AMPK β2 were treated with varying concentrations of compound 1 for 30 mins at 37 C in the presence or absence of 11 μM of compound 2. Phosphorylation of ACC was assessed using the HTRF Cisbio (pACC kit). Results are displayed as the average fold increase in activation (±SEM) of 3 independent experiments.

Compound 1: indirect activator (Urolithin B) 3-hydroxy-6H-benzo[c]chromen-6-one; CAS: 1139-83-9

Compound 2: direct activator (Benzocoumarin) 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b,d]pyran-6-one

### EXAMPLES

### Example 1

Compound 1 Increases the Phosphorylation of the AMPK Substrate, Acetyl-CoA Carboxylase (ACC), in U-2 OS Flp-In T-REx Mammalian Cells.

Natural compounds typically activate AMPK almost exclusively through their ability to interfere with ATP production of the cell, typically by inhibiting mitochondrial respiration. As a consequence, this perturbs the adenine nucleotide levels within the cell and leads to activation of AMPK through AMP and ADP binding to the AMPK γ subunit. This mechanism of AMPK activation has been termed “indirect” due to the fact that natural compounds do not directly bind to AMPK to achieve activation. In contrast, AMPK can be “directly” activated by binding of small molecules to the allosteric drug and metabolite (ADaM) binding site formed at the interface between the AMPK α subunit kinase domain and the AMPK β subunit.

We have now identified a new benzocoumarin compound, compound 2, which directly binds to the ADaM-site and activates AMPK. We achieved synergistic activation of AMPK by treating cells with a combination of an indirect benzocoumarin activator (compound 1) and this newly identified direct benzocoumarin activator (compound 2).

First, we monitored the activation of AMPK within cells using the indirect activator, compound 1. U-2 OS Flp-In T-REx cells were seeded at 50 K in a 96-well plate and left overnight at 37 C in DMEM GlutaMAX (Thermo Fisher Scientific) supplemented with 10% (vol/vol) FBS and 100 U/ml penicillin G, and 100 μg/ml streptomycin. Cells were treated for 30 mins with varying concentrations of Compound 1 in media lacking FBS and then cells were lysed in 50 μl of Cisbio lysis buffer #1 supplemented with blocking solution as per the manufacturer's protocol (Cisbio). Cells were lysed for 30 mins at room temperature before 16 μl of lysate was incubated with 4 μl of the HTRF antibodies (1:40 dilution of the acceptor and donor (p)ACC antibodies, as per the manufacturers protocol). Lysates were incubated overnight with the antibodies before the 665 nm/620 nm ratio was determined using a MolecularDevices i3 plate reader (with a HTRF cartridge add-on).

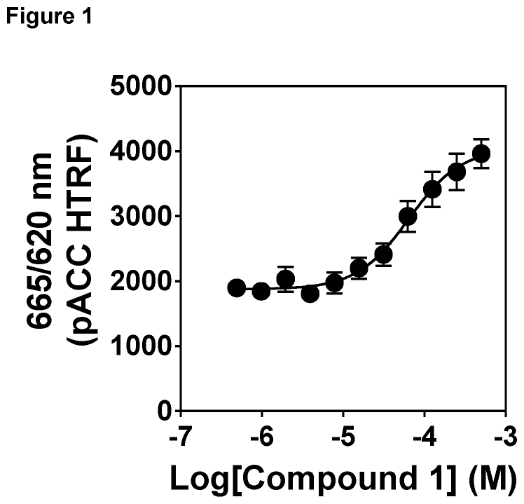

FIG. 1 shows that using the pACC HTRF assay kit (Cisbio), Compound 1 increased the phosphorylation of the AMPK substrate, ACC, in a dose-dependent manner in U-2 OS Flp-In T-REx mammalian cells. Phosphorylation of ACC is extensively used as a cellular read-out of AMPK activity.

### Example 2

Compound 2 Improves the Dose-Response Curve of Compound 1 for the Phosphorylation of the AMPK Substrate, Acetyl-CoA Carboxylase (ACC), in U-2 OS Flp-In T-REx Mammalian Cells.

To investigate whether treatment of compound 1 with our new identified direct benzocoumarin AMPK activator, compound 2, could improve the activation of AMPK, cells were treated for 30 mins with varying concentrations of compound 1 in the presence or absence of 11 μM compound 2. Cells were cultured and analysed according to example 1.

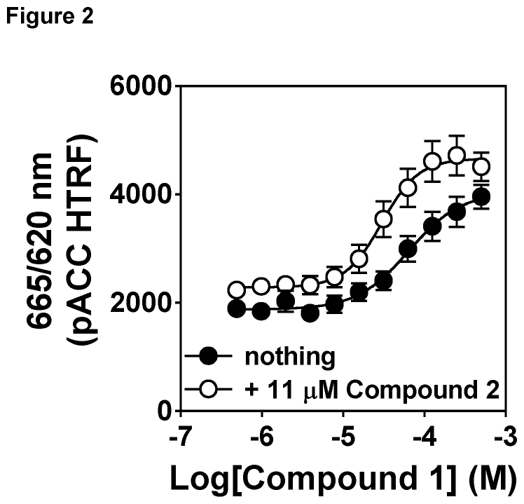

FIG. 2 shows that using the pACC HTRF assay kit (Cisbio), compound 1 increases the phosphorylation of the AMPK substrate, ACC, in a dose-dependent manner in U-2 OS Flp-In T-REx mammalian cells. Surprisingly, in cells treated with compound 1 in the presence of a fixed concentration of compound 2 showed a left-shift in the dose-response curve. This suggests that there is an improved ability of compound 1 to activate AMPK when compound 2 is present. This synergistic activation of AMPK was achieved when using two AMPK activators with differing mechanisms of action, namely, one directly and the other indirectly activating AMPK.

### Example 3

Compound 2 does not Improve the Dose-Response Curve of Compound 1 in Cells Expressing AMPK Complexes Containing a Mutation at the Allosteric Drug and Metabolite (ADaM) Site in Cells (S108A).

We investigated whether the ability of compound 2 to improve the activation profile of compound 1, was dependent on the ability of compound 2 to bind to the ADaM-site of AMPK. Interference of the regulation at the ADaM-site can be induced by either introducing a mutation at site serine 108 within the β subunit of the ADaM-site, or by switching the β isoform from β1 to β2. Activation of the β2 subunit isoform weakens or abolishes the ability of ADaM-site activators to stimulate AMPK.

AMPKβ1/β2 double knockout U-2 OS Flp-In™ T-Rex™ cell lines were generated by Horizon Discovery (Cambridge, UK). Cells were genotyped and analysed by Western blotting to confirm that there was a complete knockout of AMPKβ1/β2. We took these AMPKβ1/β2 double knockout cells, and re-introduced the expression of human β1 wild-type (WT) or a β1 Serine 108 to alanine mutation (S108A). This was achieved using the Flp-In™ system (Invitrogen) present in this cell line and stable cells expressing β1 WT or a β1 S108A mutant were generated according to the manufacturers' protocols. Re-expression of the β1 subunit was confirmed by Western blot analysis.

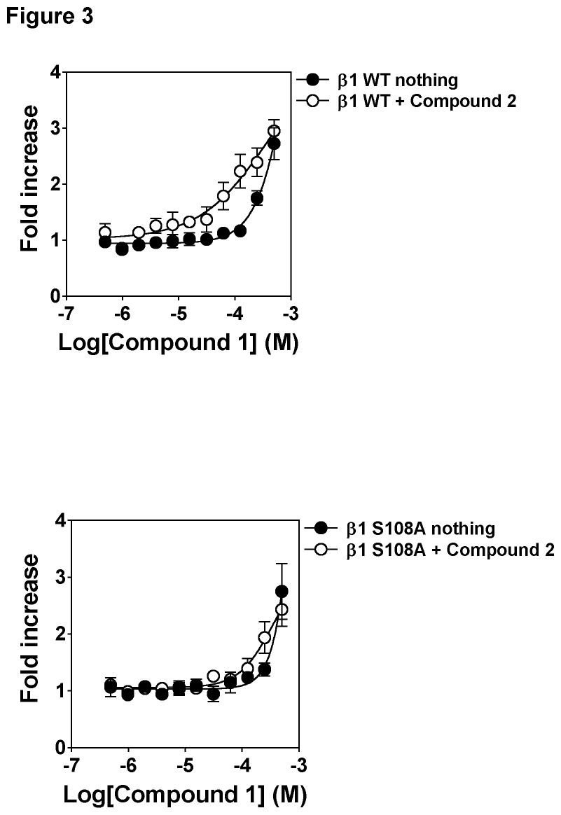

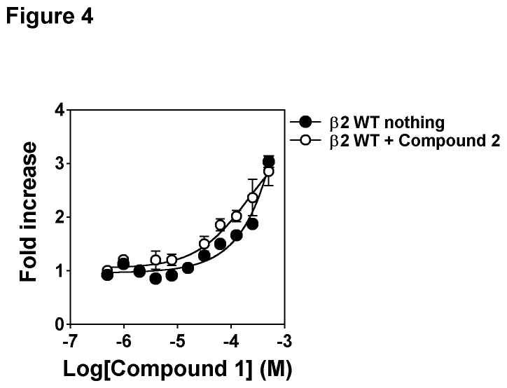

Cells stably expressing β1 WT or a β1 S108A mutant were treated with varying concentrations of Compound 1 in the presence or absence of a fixed concentration (11 μM) of compound 2. Cells lysates were subjected to the pACC HTRF (Cisbio) assay to determine the level of phosphorylation of the AMPK substrate, ACC, in cell lysates, as in example 1. As shown in FIG. 3, Compound 1 was able to dose-dependently increase pACC in cells stably expressing the β1 WT subunit. Compound 2 was capable of causing a left-shift and improvement in the ability of compound 1 to activate AMPK in β1 WT cells. In contrast, Compound 2 was not able to cause a left-shift and improvement in the dose-response curve of compound 1 in cells expressing the β1 S108A mutant. This suggests that the ability of compound 2 to improve the activation of compound 1, is mediated by its ability to bind to the ADaM-site of AMPK.

### Example 4

In Cells, Compound 2 does not Improve the Dose-Response of Compound 1 to Activate AMPK Complexes Containing the β2 Isoform Subunit.

We took AMPKβ1/β2 double knockout cells, and re-introduced the expression of the human β2 WT isoform. Re-expression of the β2 subunit was confirmed by Western blot analysis and were shown to be expressed to a similar extent. Cells stably expressing β2 WT were treated with varying concentrations of Compound 1 in the absence or presence of a fixed concentration (11 μM) of compound 2. Cell lysates were subjected to the pACC HTRF (Cisbio) assay to determine the level of phosphorylation of the AMPK substrate, ACC, in cell lysates, as in Example 1. As shown in FIG. 4, Compound 2 did not improve the dose-response curve of compound 1 in cells expressing the β2 WT isoform. Direct activation AMPK by binding to the ADaM site was impaired or abolished in β2-containing complexes in vitro and in cells. The inability of compound 2 to improve the activation of AMPK by compound 1, was consistent with the knowledge we have generated showing the compound 2 binds to the ADaM-site of AMPK. Taken together, we showed that in cells, Compound 2 binding to the ADaM-pocket, and indirect activation of AMPK (compound 1) result in synergistic activation of AMPK. Low doses of compound 1 in combination with compound 2 could activate AMPK. This demonstrated the advantage of combining two AMPK activators with different modes of action.

1. Combination of a direct AMPK activator compound, which binds directly to at least one alpha, beta or gamma subunit of AMPK; and an indirect AMPK activator compound, which does not bind directly to AMPK but alters the nucleotide status of the cell by lowering ATP in the cell and increasing AMP/ADP, to activate AMPK via the gamma-subunit; wherein the direct AMPK activator compound has the general formula I 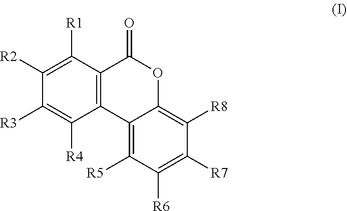
wherein R1, R2, R3, R4, R5, R6, R7, and R8 are each independently selected from the group consisting of H; CH₃; CH₂OH; CHO; COOH; OH; OCH₃; CO—(CH₂)₂—CH₃; O—CO—CH₃; a halogen; O-glycoside; C-glycoside; acylated O-glycoside; acylated C-glycoside; sulfated O-glycoside; sulfated C-glycoside; and a sulfate; and the indirect AMPK activator compound has the general formula VII 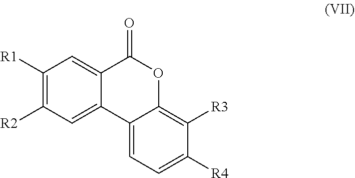
wherein R1, R2, R3, and R4 are each independently selected from the group consisting of OH; OCH₃; O-glycoside; C-glycoside; acylated O-glycoside; acylated C-glycoside; sulfated O-glycoside; sulfated C-glycoside; a sulfate; for use in the activation of AMPK.

2. Combination according to claim 1 for use in the activation of AMPK wherein said direct AMPK activator compound is a compound of Formula II 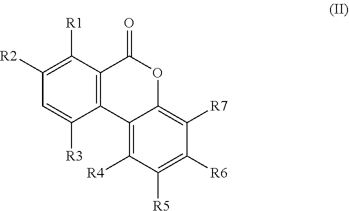
wherein R1, R2, R3, R4, and R5 are each independently selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; C1 to C20 alkyl; R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; and/or a derivative or analogue thereof, for use in the activation of AMPK.

3. Combination according to claim 1 for use in the activation of AMPK wherein said direct AMPK activator compound is a compound of Formula III 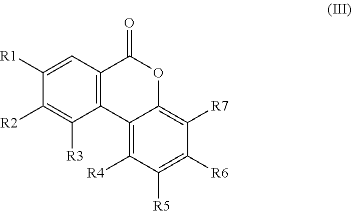
wherein R1, R2, R3, R4, and R5 are each independently selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; R6, and R7 are each independently H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; and/or a derivative or analogue thereof, for use in the activation of AMPK.

4. Combination according to claim 1 for use in the activation of AMPK wherein said direct AMPK activator compound is a compound of Formula IV 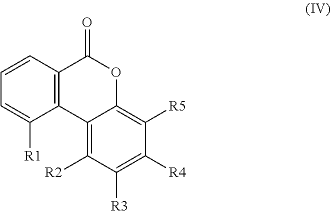
wherein R1, R2, and R3 are each independently selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; R4, and R5 are each independently selected from the group consisting of H, OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; and/or a derivative or analogue thereof, for use in the activation of AMPK.

5. Combination according to claim 1 for use in the activation of AMPK wherein said direct AMPK activator compound is a compound of Formula V 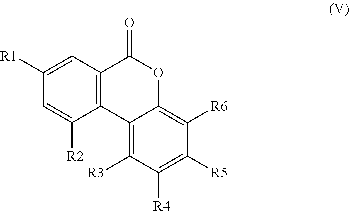
wherein R1, R2, R3, and R4 are each independently selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; R5 and R6 are each independently H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; a carboxylic acid; an ester; a primary, secondary, or tertiary amine; a primary or secondary amide; a cyano; a nitro; a sulfonate; a sulfate; and/or a derivative or analogue thereof, for use in the activation of AMPK.

6. Combination according to claim 1 for use in the activation of AMPK, wherein said direct AMPK activator compound is
3,10-Dihydroxy-8-methoxy-6H-benzo[c]chromen-6-one; 6H-Dibenzo[b,d]pyran-6-one,
3,10-Dihydroxy-8-methoxy; 3,10-Dihydroxy-8-methoxy-6H-dibenzo[b,d]pyran-6-one.

7. Combination according to claim 1 for the activation of AMPK wherein the indirect AMPK activator is a compound of Formula VII selected from the group consisting of:

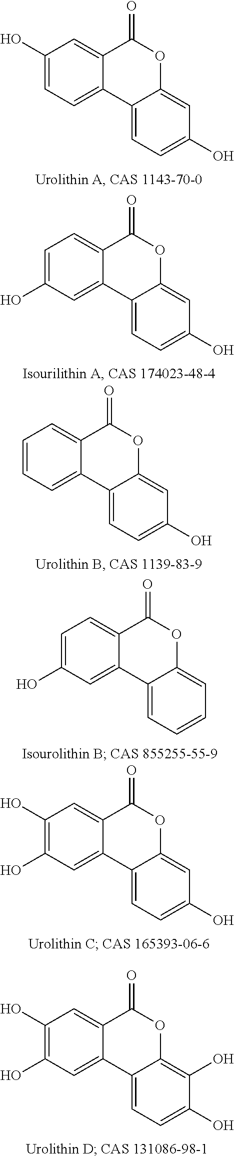

8. Combination according to claim 1 for the activation of AMPK wherein the indirect AMPK activator is Urolithin B.

9. Combination according to claim 1 for the activation of AMPK wherein the direct AMPK activator is selected from the group consisting of 3,10-Dihydroxy-8-methoxy-6H-benzo[c]chromen-6-one; 6H-Dibenzo[b,d]pyran-6-one, 3,10-Dihydroxy-8-methoxy; 3,10-Dihydroxy-8-methoxy-6H-dibenzo[b, d]pyran-6-one and the indirect AMPK activator is Urolithin B.

10. A method according to claim 1 for the activation of AMPK to treat or prevent a condition, disorder, or disease selected from the group consisting of cardiometabolic health, obesity, type 2 diabetes, non-alcoholic fatty liver disease, cardiovascular disease, and/or cancer comprising administering to a subject in need of same a combination of a direct AMPK activator compound, which binds directly to at least one alpha, beta or gamma subunit of AMPK; and an indirect AMPK activator compound, which does not bind directly to AMPK but alters the nucleotide status of the cell by lowering ATP in the cell and increasing AMP/ADP, to activate AMPK via the gamma-subunit wherein the direct AMPK activator compound has the general formula I 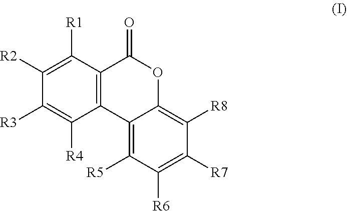
wherein R1, R2, R3, R4, R5, R6, R7, and R8 are each independently selected from the group consisting of H; CH, CH₂OH; CHO; COOH; OH; OCH₃; CO—(CH₂)₂—CH₃; O—CO—CH₃, a halogen; O-glycoside; C-glycoside; acylated O-glycoside; acylated C-glycoside; sulfated O-glycoside; sulfated C-glycoside; and a sulfate; and the indirect AMPK activator compound has the general formula VII 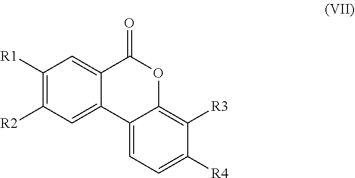
wherein R1, R2, R3, and R4 are each independently selected from the group consisting of OH; OCH₃; O-glycoside; C-glycoside; acylated O-glycoside; acylated C-glycoside; sulfated O-glycoside; sulfated C-glycoside; a sulfate; for use in the activation of AMPK.

11. Method according to claim 10 for the activation of AMPK, wherein the subject is a human.

12. Method according to claim 10, wherein the activation of AMPK is in muscle, liver and/or kidney tissues.

13. Method according to claim 1, wherein the compounds are obtained from a plant or plant extract.

14. (canceled)

15. Combination according to claim 1 wherein said combination is formulated as a food, beverage, or dietary supplement.

16. Combination according to claim 1 wherein said combination is formulated as a pharmaceutical product.

17. (canceled)

18. A method of treatment of a condition, disorder, or disease related to cardiometabolic health, obesity, type 2 diabetes, non-alcoholic fatty liver disease, cardiovascular disease, and/or cancer comprising administration of the combination of a direct AMPK activator compound, which binds directly to at least one alpha, beta or gamma subunit of AMPK, and an indirect AMPK activator compound, which does not bind directly to AMPK but alters the nucleotide status of the cell by lowering ATP in the cell and increasing AMP/ADP, to activate AMPK via the gamma-subunit; wherein the direct AMPK activator compound has the general formula I 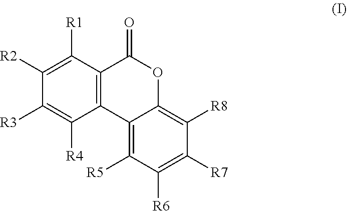
wherein R1, R2, R3, R4, R5, R6, R7, and R8 are each independently selected from the group consisting of H; CH₃; CH₂OH, CHO; COOH; OH; OCH₃; CO—(CH₂)₂—CH₃; O—CO—CH₃; a halogen; O-glycoside; C-glycoside; acylated O-glycoside; acylated C-glycoside; sulfated O-glycoside; sulfated C-glycoside; and a sulfate; and the indirect AMPK activator compound has the general formula VII 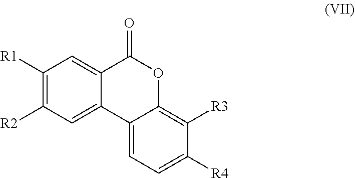
wherein R1, R2, R3, and R4 are each independently selected from the group consisting of OH; OCH₃; O-glycoside; C-glycoside; acylated O-glycoside; acylated C-glycoside; sulfated O-glycoside; sulfated C-glycoside; a sulfate; for use in the activation of AMPK to a human in need of same.
